(19) United States
(12) Patent Application Publication
(10) Pub. No.: US 2017/0071868 A1
(43) Pub. Date: Mar. 16, 2017

(54) PoP NANOCOMPOSITE STRUCTURES AND METHODS OF TRANSFERRING DRUGS USING THE SAME

(71) Applicant: SNU R&DB FOUNDATION, Gwanak-gu Seoul (KR)

(72) Inventors: Seung-Ryeoul Paik, Bucheon-si Gyeonggi-do (KR); Daekyun Lee, Yongin-si Gyeonggi-do (KR)

(21) Appl. No.: 15/123,400

(22) PCT Filed: Sep. 22, 2014
(86) PCT No.: PCT/KR2014/008774

§ 371 Date: Sep. 2, 2016

(30) Foreign Application Priority Data
Mar. 4, 2014 (KR) 10-2014-0025295

(51) Int. Cl. A61K 9/51

(52) U.S. Cl. CPC A61K 9/5169; A61K 9/5115

(57) ABSTRACT

A PoP nano-composite structure includes a first nano-particle containing a drug therein, and second nano-particles that surround a surface of the first nano-particle and are coated by a protein alpha-synuclein. Combination of the nano-particles and release of the drug can be controlled by the protein alpha-synuclein.

### BACKGROUND

1. Field

The present inventions relates to a PoP nano-composite structure and a method of transferring drugs using the same. More particularly, the present inventions relate to a PoP nano-composite structure including protein alpha-synuclein and a method of transferring drugs using the same.

2. Description of the Related Art

A porous silica nano-particle contains a lot of pores in a single particle. Thus, various materials may be stored in the pores. Recently, research and development are being conducted for using the porous silica nano-particle as a drug transfer agent that transfers a drug in a living body.

In order to commercialize the porous silica nano-particle as the drug transfer agent, improvement of biocompatibility, operation for controlling open and close of a pore for releasing a drug in a living body, improvement of transfer efficiency or the like are necessary.

For example, Patent Document 1 discloses mesoporous silica containing a drug is complexified in an inorganic powder to control releasing a drug.

[Patent Document 1] Korean Patent Publication No. 2009-0088614 (2009 August 20)

### SUMMARY

The present invention purposes to provide a PoP nano-composite structure having superior drug transfer efficiency.

The present invention also purposes to provide a method for transferring a drug using the PoP nano-composite structure having superior drug transfer efficiency.

The present invention should not be construed as limited to the exemplary embodiments set forth herein, and many modifications are possible in the exemplary embodiments without materially departing from the novel teachings and advantages of the present invention.

A PoP nano-composite structure according to an exemplary embodiment includes a first nano-particle containing a drug therein, and second nano-particles that surround a surface of the first nano-particle and are coated by a protein alpha-synuclein.

In an exemplary embodiment, the first nano-particle may include a porous silica nano-particle.

In an exemplary embodiment, the drug may be contained in a porous of the porous silica nano-particle.

In an exemplary embodiment, the pore may be closed by the second nanoparticles.

In an exemplary embodiment, the second nano-particles may include a gold nano-particle.

In an exemplary embodiment, alpha-synuclein of the protein alpha-synuclein may be a mutant by cysteine replacement.

In an exemplary embodiment, the protein alpha-synuclein may include a Y136C mutant.

According to a method for transferring a drug according to an exemplary embodiment, a drug is put in a pore of a first nano-particle. Second nano-particles coated by a protein alpha-synuclein are manufactured. The second nano-particles are adhered to a surface of the first nano-particle to form a PoP nano-composite structure. The protein alpha-synuclein is denatured to release the drug.

In an exemplary embodiment, the first nano-particle may include a porous silica nano-particle, and the second nano-particles may include a gold nano-particle.

In an exemplary embodiment, the PoP nano-composite structure may be formed in an acidic condition.

In an exemplary embodiment, the PoP nano-composite structure may be formed in a buffer solution having pH of 4 to 7.

In an exemplary embodiment, the protein alpha-synuclein may be treated with an ion to release the drug.

In an exemplary embodiment, the protein alpha-synuclein may be treated with a solution including at least one ion selected from the group consisting of calcium ion, copper ion, magnesium ion and iron ion.

In an exemplary embodiment, the second nano-particles may close the pore of the first nano-particle, and the pore may be opened by denaturation of the protein alpha-synuclein.

According to exemplary embodiments, a porous silica nano-particle containing a drug may be coated by, for example, a gold nano-particle coated by alpha-synuclein. The gold nano-particle may be self-assembled on the porous silica nano-particle because alpha-synuclein can adhere to a hydrophilic surface. Thus, a pore of the porous silica nano-particle may be easily closed to store the drug. Furthermore, release of the drug may be easily controlled by structural change of alpha-synuclein due to ligand combination.

### BRIEF DESCRIPTION OF THE DRAWINGS

FIG. 1 is a view illustrating a PoP nano-composite structure according to exemplary embodiments.

FIG. 2 is a flow chart explaining a method for transferring a drug according to exemplary embodiments.

FIG. 3 is a schematic view explaining a method for transferring a drug according to exemplary embodiments.

FIG. 4 is an electron microscope image showing a gold nano-particle coated by a protein structure and manufactured by Example 1.

FIGS. 5A and 5B are graphs showing optical properties of the nano-particle coated by protein alpha-synuclein.

FIG. 6 is an electron microscope image showing the porous silica nano-particle manufacture by Example 2.

FIGS. 7A, 7B and 7C are microscopy images showing the protein nano-composite manufactured according to Example 2.

FIG. 8 is a graph showing a ratio of the gold nano-particles that are not combined with the porous silica nano-particle, depending on pH of the buffer solution.

FIG. 9 is a graph comparing drug release from a porous silica nano-particle and from protein nano-composite having a PoP structure.

FIG. 10 is a graph showing a result of Rh6G release depending on ions.

FIG. 11 is a graph showing a result of Rh6G release depending on concentration of calcium ion.

### DETAILED DESCRIPTION

Example embodiments of the present invention are described more fully hereinafter with reference to the accompanying drawings.

The present invention may, however, be embodied in many different forms and should not be construed as limited to the example embodiments set forth herein. Rather, these example embodiments are provided so that this description will be thorough and complete, and will fully convey the scope of the present inventive concept to those skilled in the art.

The terminology used herein is for the purpose of describing particular example embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

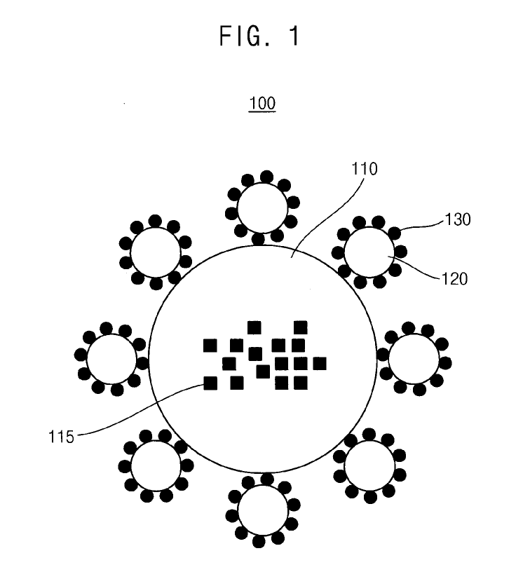

FIG. 1 is a view illustrating a PoP nano-composite structure according to exemplary embodiments.

Referring to FIG. 1, a PoP nano-composite structure 100 according to exemplary embodiments may include a first nano-particle 110 and second nano-particles 120 surrounding a surface of the first nano-particle 110. A surface of the second nano-particles 120 may be coated by a Alpha-synuclein may be adhered. A drug 115 may be contained in or inserted into the first nano-particle 110.

The first nano-particle 110 may have a porous structure. According to exemplary embodiment, the first nano-particle 110 may include a porous silica nano-particle. The porous silica nano-particle may be manufactured by a sol-gel process. For example, the porous silica nano-particle may be manufactured by Stober method using a base alcohol solution and a silicon alkoxylate such as tetraethoxysilane (TEOS).

For example, the first nano-particle 110 may include a pore having a diameter of about 2 nm to about 4 nm. The drug 115 may be contained in or inserted into the pore. The drug 115 is not limited to specific materials, and various drugs such as antibiotics, carcinostatis substances, analgesics, antiphlogistics or the like may be contained in or inserted into the pore. For example, the first nano-particle 110 may have a diameter of about 100 nm to about 200 nm.

According to exemplary embodiments, the second nano-particle 120 may include a gold nano-particle. For example, the second nano-particle 120 may have a diameter of about 5 nm to about 20 nm.

A surface of the second nano-particle 120 may be coated by the protein alpha-synuclein 130. According to exemplary embodiments, the protein alpha-synuclein 130 may include a cystein mutant of alpha-synuclein. Alpha-synuclein may have various types such as oligomer, fibrosum or the like, and may coat the second nano-particle 120.

Alpha-synuclein is an acidic protein including 140 amino acids, and includes an amphipathic N-terminal, an acidic C-terminal and a central portion, which is a hydrophobic region. Alpha-synuclein may be adhered to a hydrophilic surface in an acidic condition. Thus, alpha-synuclein may be adhered to a surface of the first nano-particle 110 having a hydrophilic surface, and may induce the second nano-particle 120 to be self-assembled adjacent to the first nano-particle 110.

Thus, a PoP (particles-on-a-particle) structure having the second nano-particles 120 integrated on the first nano-particle 110, for example, with a raspberry shape, may be obtained.

In an exemplary embodiment, the protein alpha-synuclein 130 may include a cystein mutant of alpha-synuclein. For example, amino acids of alpha-synuclein in protein alpha-synuclein 130 may be partially replaced by cysteine, or cysteine may be inserted into the alpha-synuclein.

According to exemplary embodiments, the protein alpha-synuclein 130 may be alpha-synuclein Y136C mutant, of which a C-terminal is replaced by cysteine. The alpha-synuclein Y136C mutant 130 may be easily conjugated with the second nano-particle 120 by a mercapto group (—SH) of a terminal. The gold nano-particle coated by alpha-synuclein may be adhered to the surface of the first nano-particle in acidic pH. Thus, the PoP structure may be obtained by the protein alpha-synuclein 130.

According to exemplary embodiments, the second nano-particle 120 coated by protein alpha-synuclein 130 may close the pore of the first nano-particle 110. Thus, the PoP nano-composite structure 100 may be delivered into a living body without leakage of the drug therein.

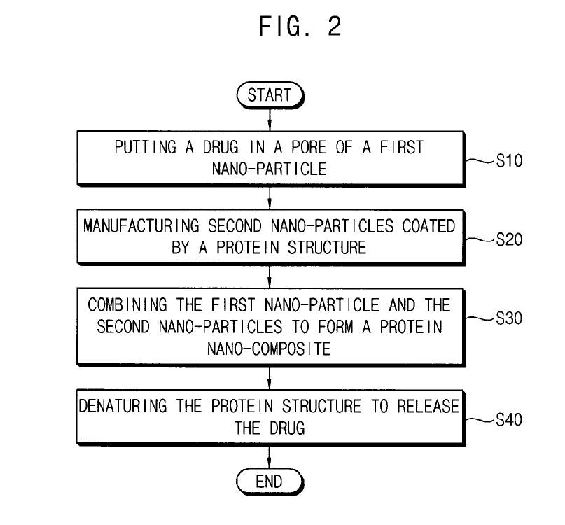

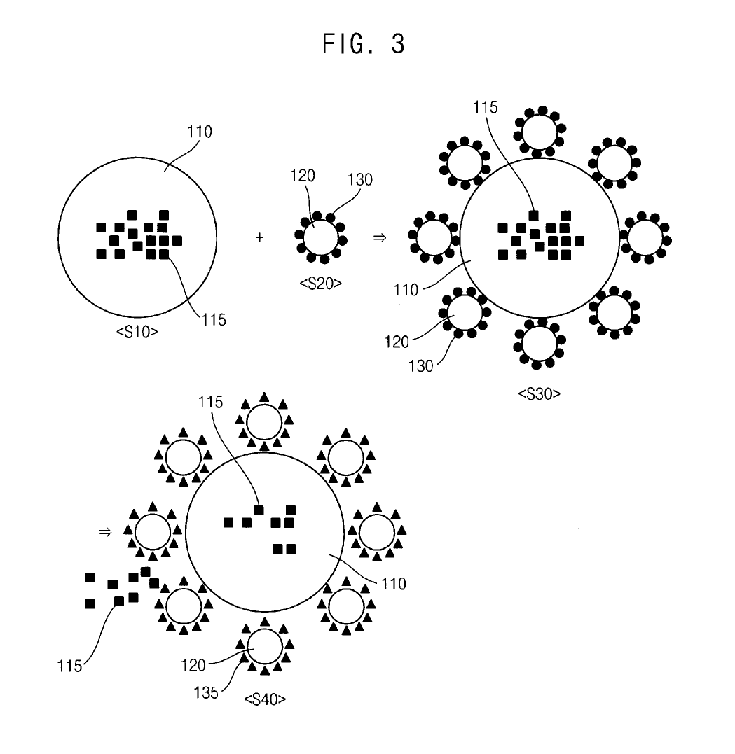

FIG. 2 is a flow chart explaining a method for transferring a drug according to exemplary embodiments. FIG. 3 is a schematic view explaining a method for transferring a drug according to exemplary embodiments.

Referring to FIGS. 2 and 3, a drug 115 is put in a pore of a first nano-particle 110 (S10) after the first nano-particle 110 is manufactured.

According to exemplary embodiments, the first nano-particle 110 may be a porous silica nano-particle manufactured by Stober method using a silicon alkoxylate such as tetraethoxysilane (TEOS). In an exemplary embodiment, the porous silica nano-particle may be synthesized by using a surfactant such as cetyltrimethylammonium for a mold. The porous silica nano-particle may be dipped into a solution including the drug 115 so that the drug 115 may be contained in the pore.

A second nano-particle 120 coated by protein alpha-synuclein 130 is manufactured (S20).

According to exemplary embodiments, a gold nano-particle may be used for the second nano-particle 120, and alpha-synuclein Y136C mutant may be used for the protein alpha-synuclein 130. For example, gold nano-particles may be dispersed in a solution including alpha-synuclein Y136C mutant so that a surface of each gold nano-particles may be coated by alpha-synuclein.

In an exemplary embodiment, a C-terminal of alpha-synuclein may be replaced by cysteine so that the alpha-synuclein may be conjugated with the gold nano-particles by a mercapto group.

The second nano-particles 120 and the first nano-particle 110 are combined with each other to form a PoP nano-composite structure (S30).

According to exemplary embodiments, the PoP nano-composite structure may have a PoP structure wherein the second nano-particles 120 are self-assembled on the surface of the first nano-particle 110. For example, the first nano-particle 110 and the second nano-particles 120 coated by protein alpha-synuclein 130 may react with each other in a predetermined buffer solution to form the PoP nano-composite structure.

According to exemplary embodiments, the buffer solution may be an acidic solution may be an acidic solution having pH between about 4 to about 7. When pH of the buffer solution is less than about 4, acidity excessively increases so that a structure of alpha-synuclein or a surface or a structure of the first nano-particle 110 may be damaged. When pH of the buffer solution is more than about 7, a combination ratio of the first nano-particle 110 and the second nano-particle 120 may be reduced. In the above pH range, the second nano-particles 120 may be assembled on the surface of the first nano-particle 110 to substantially form a singly-layered structure.

As explained in the above, the protein alpha-synuclein 130 that coats the second nano-particle 120 may have an N-terminal of a protein exposed to exterior. Thus, the protein alpha-synuclein 130 may be easily adhered to or conjugated with the first nano-particle 110 having an inorganic silica surface through structural change in acidic pH.

Furthermore, the pore of the first nano-particle 110 may be closed by the second nano-particles 120. Thus, leakage of a drug may be prevented.

The drug in the pore of the first nano-particle 110 is released by inducement of structural change of the protein alpha-synuclein 130 (S40).

According to exemplary embodiments, alpha-synuclein may be denaturation-treated to change a chemical structure of the alpha-synuclein. Thus, the protein alpha-synuclein 130 may be changed into a denatured protein 135 so that location or phase of the second nano-particle 120 blocking the pore of the first nano-particle 110. Thus, the pore that was blocked is opened to induce release of the drug.

Examples of a treatment for structural change of the protein may include ion-treatment, ligand-treatment or external stimulation such as light, temperature, change of pH or the like. In an exemplary embodiment, the PoP nano-composite structure and/or the protein alpha-synuclein 130 may be treated with a metal cation to form the denatured protein 135, of which structural properties are changed, thereby inducing release of the drug. For example, an adhesion structure of the protein alpha-synuclein 130 may be changed by the ion treatment to form the denatured protein 135.

Examples of the metal cation may include calcium ion (Ca2+), copper ion (Cu2+), magnesium ion (Mg2+) or iron ion (Fe2+). These can be used each alone or in a combination thereof. When the ion-treatment is performed, a release amount of the drug may be adjusted by varying concentration of ions.

In an exemplary embodiment, a dye such as phthalocyanine or a biotic hormone such as dopamine may be used for the ligand-treatment.

According to exemplary embodiments, porous inorganic nano-particles for transferring a drug are coated or conjugated with alpha-synuclein. Thus, a biocompatibility and a durability such as half-life of a drug transfer agent may be increased. Furthermore, a pore of a first nano-particle such as a porus silica nano-particle may be closed or blocked by second nano-particles coated by protein alpha-synuclein to prevent leakage of a drug contained in the pore.

Additionally, release of the drug may be effectively controlled by ion-treatment, ligand-treatment and/or external stimulation.

Hereinafter, a PoP nano-composite structure and a method of transferring drugs using the same according to exemplary embodiments will be explained with reference to particular examples.

### Example 1

### Manufacturing a Gold Nano-Particle Coated by Protein Alpha-Synuclein

Alpha-synuclein cDNA, of which tyrosine of C-terminal is replaced by cysteine, was manufactured by using a commercialized site-directed mutagenesis kit. The cDNA was inserted into a protein expression vector. The vector having the cDNA inserted thereinto was injected into Escherichia coli to induce expression of a protein mutant. The expressed alpha-synuclein mutant was refined by ion exchange chromatography and gel permeation chromatography. As a result, alpha-synuclein Y136C mutant, of which a C-terminal was replaced by cysteine, was obtained. A surface of a gold nano-particle was coated with the alpha-synuclein Y136C mutant.

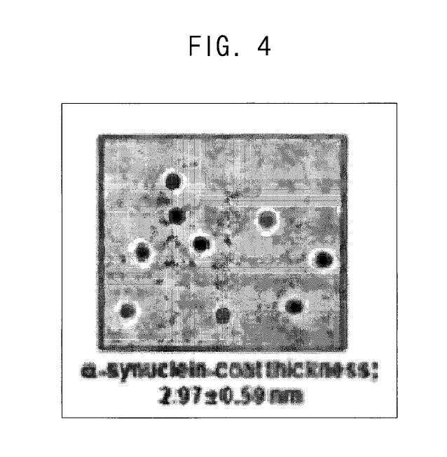

FIG. 4 is an electron microscope image showing a gold nano-particle coated by a protein structure and manufactured by Example 1.

Referring to FIG. 4, it can be noted that alpha-synuclein formed a coating layer having substantially uniform thickness adjacent the gold nano-particle.

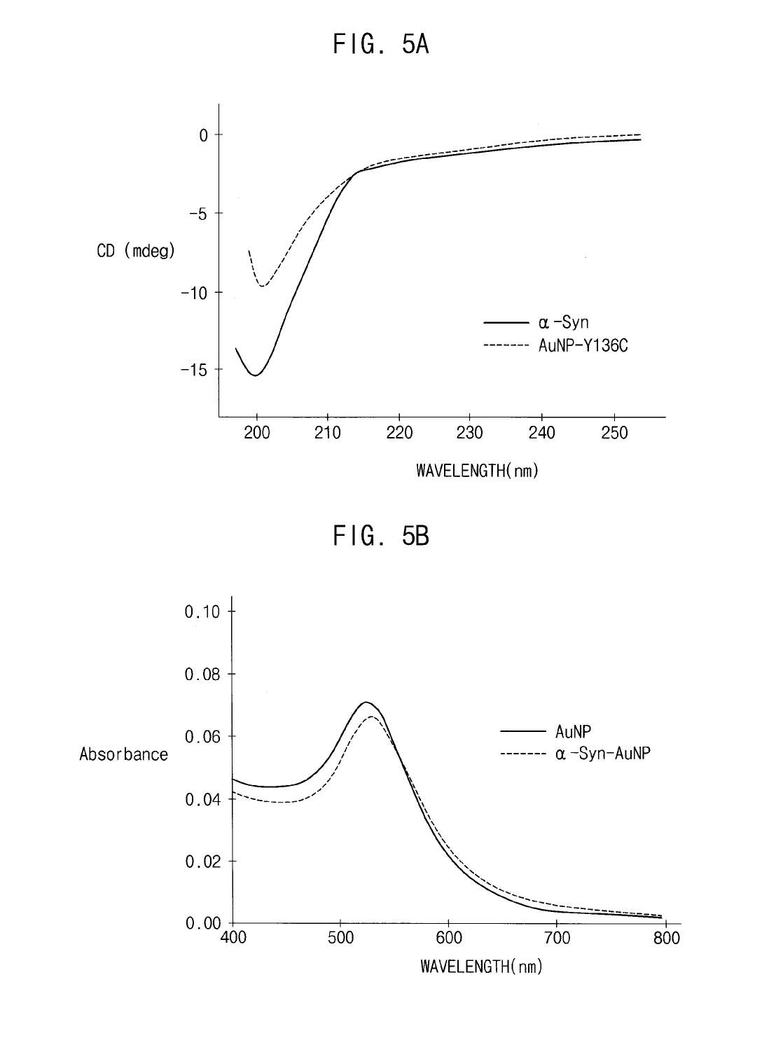

FIGS. 5A and 5B are graphs showing optical properties of the nano-particle coated by protein alpha-synuclein. Particularly, FIGS. 5A and 5B respectively show optical properties of the alpha-synuclein and the gold nano-particle themselves in the nano-particle coated by the protein alpha-synuclein through circular dichoroism spectroscopy and surface plasmon resonance spectroscopy.

Referring to FIG. 5A, it can be noted that similar graphs was obtained for alpha-synuclein (α-Syn) and Y136C mutant coated on a gold nano-particle (AuNP-Y136C) by circular dichoroism spectroscopy.

Referring to FIG. 5B, it can be noted that similar graphs was obtained for a gold nano-particle (AuNP) and a gold nano-particle coated by alpha-synuclein (α-Syn-AuNP) by surface plasmon resonance spectroscopy.

Thus, it can be noted that even if alpha-synuclein is coated on a gold nano-particle, their own structure and properties do not change.

### Example 2

### Manufacturing a Protein Nano-Composite Having a PoP Structure

A porous silica nano-particle was manufactured through a known Stober method using cetyltrimethylammonium, which is a surfactant having a cation, as a mold. The porous silica nano-particle had a diameter of about 100 nm and a pore size of about 2 nm to about 3 nm.

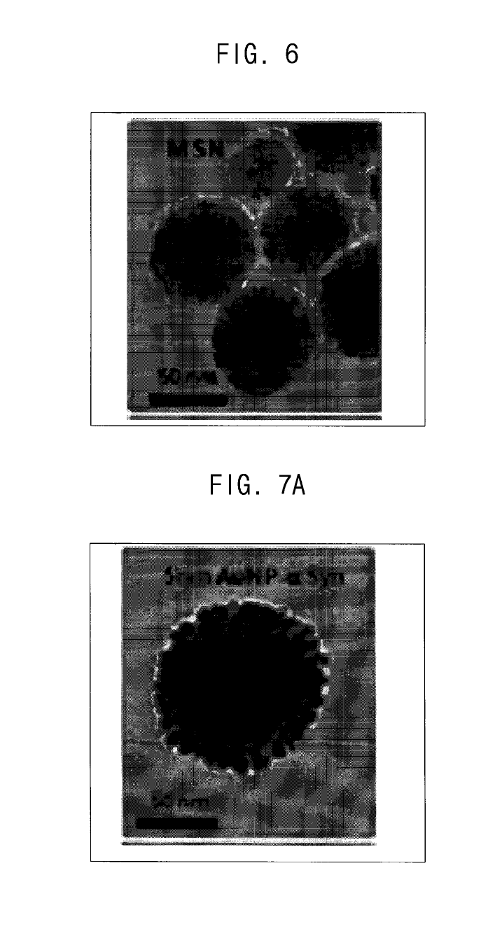

FIG. 6 is an electron microscope image showing the porous silica nano-particle manufacture by Example 2.

Thereafter, the gold nano-particle coated by alpha-synuclein and manufactured according to Example 1 and the porous silica nano-particle were put in a buffer solution having pH of about 4.4 to react for about 30 minutes. As a result, a nano-structure having the gold particles self-assembled on the porous silica nano-particle to form a PoP structure was obtained.

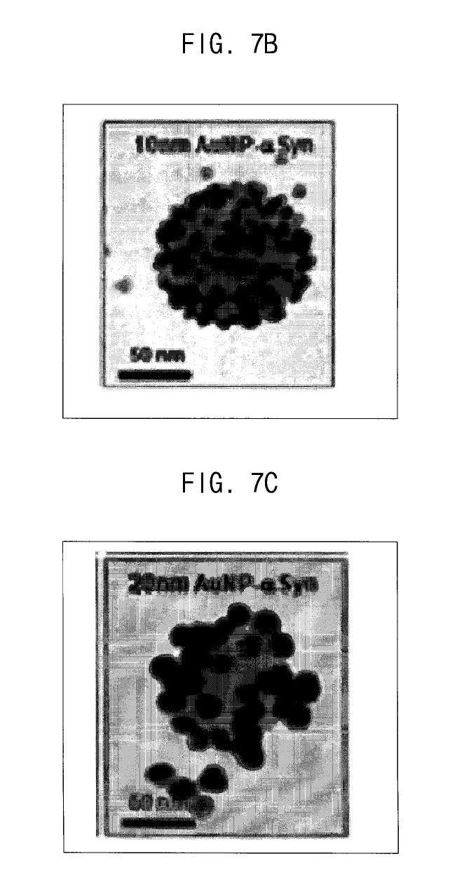

FIGS. 7A, 7B and 7C are microscopy images showing the protein nano-composite manufactured according to Example 2. Particularly, FIGS. 7A, 7B and 7C show protein nano-composites coated by gold nano-particles respectively having diameters of 5 nm, 10 nm and 20 nm.

Referring to FIGS. 7A, 7B and 7C, it can be noted that the gold nano-particles were assembled on a surface of the porous silica nano-particle with a substantial single-layered structure regardless of diameters of the gold nano-particles

Furthermore, combination extent of the porous silica nano-particle and the gold nano-particles coated by alpha-synuclein were measured according to pH.

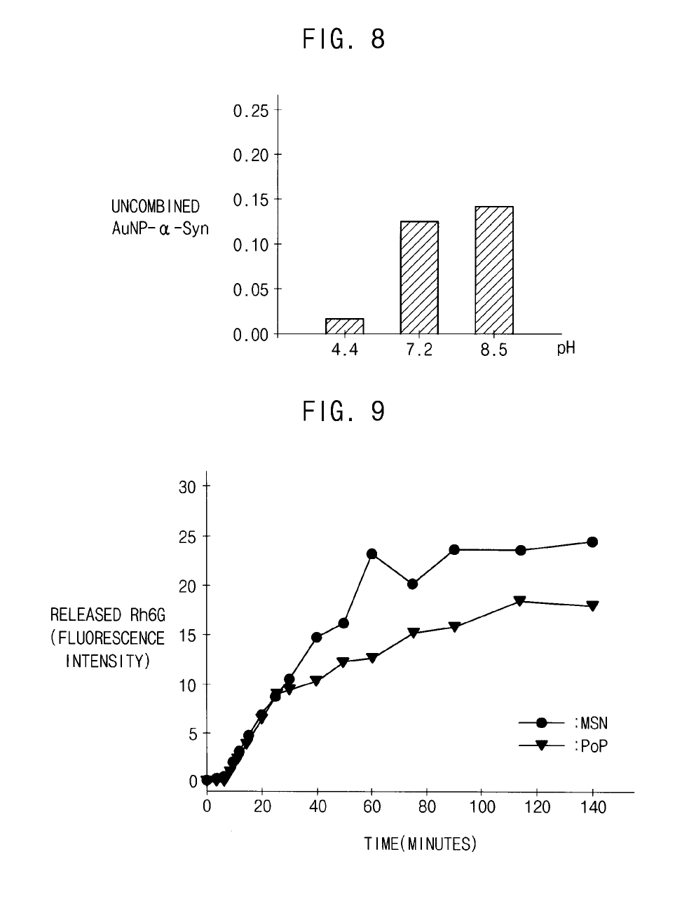

FIG. 8 is a graph showing a ratio of the gold nano-particles that are not combined with the porous silica nano-particle, depending on pH of the buffer solution.

Referring to FIG. 8, a fraction of the gold nano-particles coated by alpha-synuclein (α-Syn-AuNP) that are not combined with the porous silica nano-particle was equal to or more than about 0.15 at pH of about 8.5. The fraction was reduced to be less than about 0.15 at pH of about 7.2, and was reduced to be less than about 0.05. Thus, it can be noted that formation of the PoP nano-composite may be accelerated by adjusting pH of the buffer solution to be approximate to 4.

### Example 3

### Evaluation for Drug-Containing Ability of PoP Nano-Composite

Rhodamine 6G (Rh6G) was put in the porous silica nano-particle manufactured according to Example 2 in order to indirectly evaluate a drug-containing ability of a PoP nano-composite according to an exemplary embodiment. Thereafter, the gold nano-particle coated by alpha-synuclein Y136C mutant and the porous silica nano-particle were combined with each other to manufacture a PoP nano-composite.

The PoP nano-composite was put into a water solution and then measured if Rh6G was released therefrom, through fluorometry.

FIG. 9 is a graph comparing drug release from a porous silica nano-particle (represented by MSN) and from protein nano-composite having a PoP structure (represented by PoP).

Referring to FIG. 9, it can be noted that, after lapse of about 20 minutes, drug release speed of PoP was remarkably lower than that of MSN, which was not combined with a gold nano-particle. Furthermore, it can be noted that, after lapse of about 2 hours, drug release of PoP was not processed anymore and that PoP could contain drug more than MSN by about 30%.

### Example 4

### Evaluation for Drug-Releasing Ability of PoP Nano-Composite

The PoP nano-composite was treated with various ions in order to induce drug-release from the PoP nano-composite containing Rh6G and manufactured according to Example 3.

Particularly, solutions respectively including 2 mM of Ca²⁺, Cu²⁺, Mg²⁺, Fe³⁺, Na⁺ and K⁺ were prepared. The PoP nano-composite containing Rh6G was put into each of the solutions to react for about 10 minutes. Thereafter, release extent of Rh6G was observed.

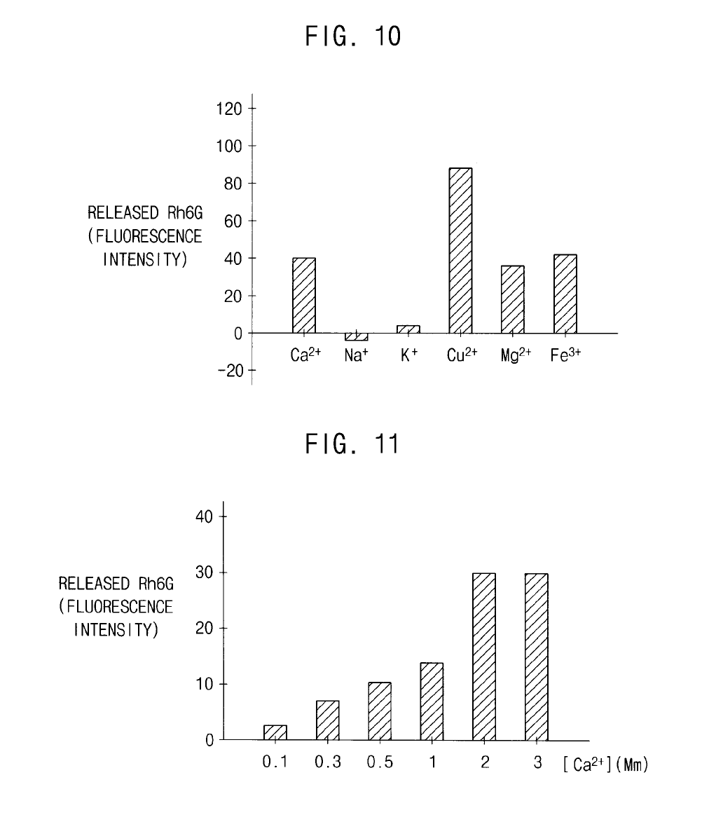

FIG. 10 is a graph showing a result of Rh6G release depending on ions.

Referring to FIG. 10, it can be noted that Rh6G was immediately released when the protein nano-composite was treated with Ca²⁺, Cu²⁺, Mg²⁺ or Fe³⁺. Especially, it can be noted that Rh6G was strongly released when the protein nano-composite was treated with Cu²⁺.

FIG. 11 is a graph showing a result of Rh6G release depending on concentration of calcium ion.

Referring to FIG. 11, it can be noted that Rh6G was released substantially proportional to concentration of calcium ion as concentration of calcium ion increased and that release of Rh6G may be stopped or reduced when concentration of calcium ion was more than a critical concentration, for example, 3 mM as shown in FIG. 11.

Thus, it can be noted that drug release amount may be controlled by adjusting concentration of an ion used for ion-treatment.

The PoP nano-composite and the method for transferring a drug according exemplary embodiments of the present invention may be used for manufacturing a drug transfer agent having a high biocompatibility and a drug transfer system. Thus, the PoP nano-composite and the method for transferring a drug may be used for transferring a biomedical agent for curing various interactable diseases, including carcinostatis substances, and for a biosensor.

The foregoing is illustrative and is not to be construed as limiting thereof. Although a few exemplary embodiments have been described, those skilled in the art will readily appreciate that many modifications are possible in the exemplary embodiments without materially departing from the novel teachings, aspects, and advantages of the invention. Accordingly, all such modifications are intended to be included within the scope of this disclosure.

What is claimed is:

1. A PoP (particles-on-a-particle) nano-composite structure comprising
a first nano-particle containing a drug therein; and
second nano-particles that surround a surface of the first nano-particle and are coated by a protein alpha-synuclein.

2. The PoP nano-composite structure of claim 1, wherein the first nano-particle comprises a porous silica nano-particle.

3. The PoP nano-composite structure of claim 2, wherein the drug is contained in a porous of the porous silica nano-particle.

4. The PoP nano-composite structure of claim 3, wherein the pore is closed by the second nanoparticles.

5. The PoP nano-composite structure of claim 1, wherein the second nano-particles comprise a gold nano-particle.

6. The PoP nano-composite structure of claim 1, wherein alpha-synuclein of the protein alpha-synuclein is a mutant by cysteine replacement.

7. The PoP nano-composite structure of claim 6, wherein the protein alpha-synuclein comprises a Y136C mutant.

8. A method for transferring a drug, the method comprising:
putting a drug in a pore of a first nano-particle;
manufacturing second nano-particles coated by a protein alpha-synuclein;
adhering the second nano-particles to a surface of the first nano-particle to form a PoP nano-composite structure; and
denaturing the protein alpha-synuclein to release the drug.

9. The method of claim 8, wherein the first nano-particle comprises a porous silica nano-particle, and the second nano-particles comprise a gold nano-particle.

10. The method of claim 8, wherein the PoP nano-composite structure is formed in an acidic condition.

11. The method of claim 10, wherein the PoP nano-composite structure is formed in a buffer solution having pH of 4 to 7.

12. The method of claim 8, wherein the protein alpha-synuclein is treated with an ion to release the drug.

13. The method of claim 12, wherein the protein alpha-synuclein is treated with a solution including at least one ion selected from the group consisting of calcium ion, copper ion, magnesium ion and iron ion.

14. The method of claim 8, wherein the second nano-particles close the pore of the first nano-particle, and the pore is opened by denaturation of the protein alpha-synuclein.
